(19) United States
(12) Patent Application Publication
(10) Pub. No.: US 2024/0366392 A1
(43) Pub. Date: Nov. 7, 2024

(54) KNEE ARTHROPLASTY DEVICES, SYSTEMS, AND METHODS

(71) Applicant: E. Marlowe GOBLE, Logan, UT (US)

(72) Inventor: E. Marlowe GOBLE, Logan, UT (US)

(21) Appl. No.: 18/656,562

(22) Filed: May 6, 2024

Related U.S. Application Data
(60) Provisional application No. 63/464,557, filed on May 6, 2023.

(51) Int. Cl. A61F 2/38

(52) U.S. Cl. CPC A61F 2/389; A61F 2/3886

(57) ABSTRACT

An implant may include at least one implant articular surface positioned on a superior side of the implant and a bone-facing surface positioned on an inferior side of the implant which may be configured to couple within a Goal Line tunnel of a tibial bone. The implant may be shaped to be at least partially received within the Goal Line tunnel of the tibial bone, and a length of the implant may be oriented antero-posteriorly along a Goal Line located within the Goal Line tunnel. The at least one implant articular surface may be configured to receive a compression load force from at least one femoral articular surface engaged therewith, and the bone-facing surface coupled within the Goal Line tunnel may be configured to transmit the compression load force to the tibial bone along the Goal Line.

### CROSS-REFERENCE TO RELATED APPLICATIONS

This application claims the benefit of U.S. Provisional Patent Application Ser. No. 63/464,557 filed on May 6, 2023, entitled “WEIGHT BEARING ON THE GOAL LINE”. The above-referenced document is hereby incorporated by reference in its entirety.

### TECHNICAL FIELD

The present disclosure relates to knee arthroplasty devices, system, and methods. More specifically, the present disclosure relates to knee arthroplasty devices, system, and methods for distributing weight bearing load forces along a Goal Line of a tibia intermediate the medial and lateral tibial condyles.

### BACKGROUND

Total Knee Arthroplasty (TKA) procedures utilize invasive surgical procedures to replace one or more diseased natural articular surfaces of a knee joint with one or more artificial articular surfaces to reduce pain and restore knee joint function.

However, these invasive surgical procedures can present unacceptable levels of risk for: morbidity, infections, implant failures (that may require one or more revision surgeries), long healing and/or rehabilitation times, increased costs, etc. These risks are especially high for older individuals experiencing knee joint diseases that are progressing toward the need for a TKA procedure at some point in the future, but who presently retain adequate knee function.

Accordingly, improved devices, systems, and methods for distributing weight bearing load forces across a knee joint (utilizing less invasive surgical techniques) in order to delay, or entirely prevent, the need for TKA procedures, would be desirable.

### BRIEF DESCRIPTION OF THE DRAWINGS

Exemplary embodiments of the present disclosure will become more fully apparent from the following description taken in conjunction with the accompanying drawings. Understanding that these drawings depict only exemplary embodiments and are, therefore, not to be considered limiting of the scope of the present disclosure, the exemplary embodiments of the present disclosure will be described with additional specificity and detail through use of the accompanying drawings in which:

FIG. 1A illustrates a top view of a resected tibial plateau, according to an example of the present disclosure;

FIG. 1B illustrates a top perspective view of the resected tibial plateau of FIG. 1A;

FIG. 1C illustrates a side view of the resected tibial plateau of FIG. 1A;

FIG. 2 illustrates a perspective anterior view a femur and a tibia placed in alignment relative to each other in partial flexion, according to an example of the present disclosure;

FIG. 3 illustrates a an anterior view of the femur and tibia of FIG. 2 placed in flexion;

FIG. 4 illustrates a perspective antero-superior view of the femur and tibia of FIG. 2 placed in full flexion with an implant inserted within a Goal Line tunnel of the tibia, according to an embodiment of the present disclosure;

FIG. 5A illustrates a top view of the implant from FIG. 4;

FIG. 5B illustrates a bottom view of the implant shown in FIG. 5A; and

FIG. 5C illustrates a side view of the implant shown in FIG. 5A.

It is to be understood that the drawings are for purposes of illustrating the concepts of the present disclosure and may not be drawn to scale. Furthermore, the drawings illustrate exemplary embodiments and do not represent limitations to the scope of the present disclosure.

### SUMMARY

The knee arthroplasty devices, systems, and methods of the present disclosure have been developed in response to the present state of the art, and in particular, in response to the problems and needs in the art that have not yet been fully solved by currently available knee arthroplasty devices, systems, and methods. In some embodiments, the knee arthroplasty devices, systems, and methods of the present disclosure may provide improved implants and surgical procedures for distributing weight bearing load forces along a Goal Line of a tibia intermediate the medial and lateral condyles.

In some embodiments, an implant may include a bone-facing surface positioned on an inferior side of the implant which may be configured to couple within a Goal Line tunnel of a tibial bone. The implant may also include at least one implant articular surface positioned on a superior side of the implant, opposite the bone-facing surface. The implant may be shaped to be at least partially received within the Goal Line tunnel of the tibial bone, and a length of the implant may be oriented antero-posteriorly along a Goal Line located within the Goal Line tunnel. The at least one implant articular surface may be configured to receive a compression load force from at least one femoral articular surface engaged with the at least one implant articular surface, and the bone-facing surface coupled within the Goal Line tunnel may be configured to transmit the compression load force to the tibial bone along the Goal Line.

In some embodiments, a majority of a width of the implant may be positionable within the Goal Line tunnel.

In some embodiments, a majority of the length of the implant may be positionable along the Goal Line within the Goal Line tunnel.

In some embodiments, at least one of: a height, a shape, and a position of the at least one implant articular surface, relative to the at least one femoral articular surface, may be selectable to receive a desired percentage range of the compression load force for transmission to the tibial bone.

In some embodiments, the desired percentage range may be 10% to 90%.

In some embodiments, the desired percentage range may be 20% to 80%.

In some embodiments, the desired percentage range may be 30% to 70%.

In some embodiments, the desired percentage range may be 40% to 60%.

In some embodiments, the desired percentage range may be configured to increase from flexion toward extension of a knee joint comprising the implant.

In some embodiments, the at least one implant articular surface may comprise a medial implant articular surface and a lateral implant articular surface. The at least one femoral articular surface may comprise a medial femoral articular surface and a lateral femoral articular surface. The medial implant articular surface may be configured to receive a first compression load force from the medial femoral articular surface that is engaged with the medial implant articular surface. The lateral implant articular surface may be configured to receive a second compression load force from the lateral femoral articular surface that is engaged with the lateral implant articular surface.

In some embodiments, the medial implant articular surface and the lateral implant articular surface may comprise concave surfaces, and the medial femoral articular surface and the lateral femoral articular surface may comprise convex surfaces.

In some embodiments, the implant may also comprise an implant ridge positioned intermediate the medial implant articular surface and the lateral implant articular surface.

In some embodiments, the implant ridge may comprise a concave curvature along the length of the implant.

In some embodiments, a tibial implant may comprise an elongate body having a length, a width, a bone-facing surface positioned on an inferior side of the elongate body configured to couple within a Goal Line tunnel of a tibial bone, and at least one implant articular surface positioned on a superior side of the elongate body, opposite the bone-facing surface. The elongate body may be positionable within the Goal Line tunnel, and the bone-facing surface of the elongate body may be couplable therein to secure the tibial implant to the tibial bone. The length of the elongate body may be oriented antero-posteriorly along a Goal Line defined within the Goal Line tunnel. The width of the elongate body may be configured to cover no more than 20% of a width of a condyle of the tibial bone.

In some embodiments, a majority of the width of the elongate body may be positionable within the Goal Line tunnel.

In some embodiments, a majority of the length of the elongate body may be positionable along the Goal Line located within the Goal Line tunnel.

In some embodiments, the at least one implant articular surface may be configured to receive a compression load force from at least one femoral articular surface that is engaged with the at least one implant articular surface. The bone-facing surface coupled within the Goal Line tunnel may be configured to transmit the compression load force to the tibial bone.

In some embodiments, at least one of: a height, a shape, and a position of the at least one implant articular surface, relative to the at least one femoral articular surface, may be selectable to receive a desired percentage range of the compression load force for transmission to the tibial bone.

In some embodiments, the desired percentage range may be 10% to 90%.

In some embodiments, the desired percentage range may be 20% to 80%.

In some embodiments, the desired percentage range may be 30% to 70%.

In some embodiments, the desired percentage range may be 40% to 60%.

In some embodiments, the desired percentage range may be configured to increase from flexion toward extension of a knee joint that comprises the tibial implant.

In some embodiments, the at least one implant articular surface may comprise a medial implant articular surface and a lateral implant articular surface. The at least one femoral articular surface may comprise a medial femoral articular surface and a lateral femoral articular surface. The medial implant articular surface may be configured to receive a first compression load force from the medial femoral articular surface that is engaged with the medial implant articular surface. The lateral implant articular surface may be configured to receive a second compression load force from the lateral femoral articular surface that is engaged with the lateral implant articular surface.

In some embodiments, the medial implant articular surface and the lateral implant articular surface may comprise concave surfaces, and the medial femoral articular surface and the lateral femoral articular surface may comprise convex surfaces.

In some embodiments, the tibial implant may also comprise an implant ridge positioned intermediate the medial implant articular surface and the lateral implant articular surface.

In some embodiments, the implant ridge may comprise a concave curvature along a length of the implant ridge.

In some embodiments, a method for installing a tibial implant within a Goal Line tunnel formed in a tibial plateau of a tibial bone may comprise: exposing the tibial plateau at a surgical site proximate the Goal Line Tunnel, inserting the tibial implant through the surgical site and into the Goal Line tunnel of the tibial plateau, and coupling a bone-facing surface of the tibial implant to the Goal Line tunnel to secure the tibial implant to the tibial bone.

In some embodiments, the method may also comprise orienting a length of the tibial implant along a Goal Line located within the Goal Line tunnel.

In some embodiments, the method may also comprise preparing the Goal Line tunnel to receive the tibial implant therein.

In some embodiments, preparing the Goal Line tunnel to receive the tibial implant therein may comprise excising an anterior cruciate ligament that is coupled to an anterior aspect of the Goal Line tunnel.

In some embodiments, preparing the Goal Line tunnel to receive the tibial implant therein may comprise widening the Goal Line tunnel to receive the tibial implant therein.

In some embodiments, the tibial implant may be inserted into the Goal Line tunnel from an anterior-to-posterior direction.

In some embodiments, the tibial implant may be inserted into the Goal Line tunnel from a posterior-to-anterior direction.

In some embodiments, a method of distributing a compression load force to a Goal Line tunnel of a tibial bone via an implant placed therein may comprise: receiving at least a portion of the compression load force at one or more implant articular surfaces positioned on a superior side of the implant, transmitting the at least a portion of the compression load force to a bone-facing surface positioned on an inferior side of the implant that is coupled within the Goal Line tunnel, and distributing the at least a portion of the compression load force through the Goal Line tunnel to the tibial bone from the implant that is coupled within the Goal Line tunnel.

In some embodiments, the at least a portion of the compression load force may be distributed through the Goal Line tunnel to the tibial bone along a Goal Line located within the Goal Line tunnel.

In some embodiments, the method may also comprise selecting at least one of: a height, a shape, and a position of the one or more implant articular surfaces, relative to one or more femoral articular surfaces, to receive a desired percentage range of the compression load force for transmission to the tibial bone through the Goal Line tunnel.

In some embodiments, the desired percentage range may be 10% to 90%.

In some embodiments, the desired percentage range may be configured to increase from flexion toward extension of a knee joint that comprises the implant.

These and other features and advantages of the present disclosure will become more fully apparent from the following description and appended claims or may be learned by the practice of the knee arthroplasty devices, systems, and methods set forth hereinafter.

### DETAILED DESCRIPTION

Exemplary embodiments of the present disclosure will be best understood by reference to the drawings, wherein like parts are designated by like numerals throughout. It will be readily understood that the components of the present disclosure, as generally described and illustrated in the drawings could be arranged and designed in a wide variety of different configurations. Thus, the following more detailed description of the embodiments of the devices, systems, and methods, as represented in the drawings, is not intended to limit the scope of the present disclosure but is merely representative of exemplary embodiments of the present disclosure.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in the drawings, the drawings are not necessarily drawn to scale unless specifically indicated.

Standard medical planes of reference and descriptive terminology are employed in this specification. While these terms are commonly used to refer to the human body, certain terms may also be applicable to physical objects in general.

A standard system of three mutually perpendicular reference planes is employed. A sagittal plane divides a body into right and left portions. A coronal plane divides a body into anterior and posterior portions. A transverse plane divides a body into superior and inferior portions. A mid-sagittal, mid-coronal, or mid-transverse plane divides a body into equal portions, which may be bilaterally symmetric. The intersection of the sagittal and coronal planes defines a superior-inferior or cephalad-caudal axis. The intersection of the sagittal and transverse planes defines an anterior-posterior axis. The intersection of the coronal and transverse planes defines a medial-lateral axis. The superior-inferior or cephalad-caudal axis, the anterior-posterior axis, and the medial-lateral axis are mutually perpendicular.

Anterior means toward the front of a body. Posterior means toward the back of a body. Superior or cephalad means toward the head. Inferior or caudal means toward the feet or tail. Medial means toward the midline of a body, particularly toward a plane of bilateral symmetry of the body. Lateral means away from the midline of a body or away from a plane of bilateral symmetry of the body. Axial means toward a central axis of a body. Abaxial means away from a central axis of a body. Ipsilateral means on the same side of the body. Contralateral means on the opposite side of the body. Proximal means toward the trunk of the body. Proximal may also mean toward a user or operator. Distal means away from the trunk. Distal may also mean away from a user or operator. Dorsal means toward the top of the foot. Plantar means toward the sole of the foot. Varus means deviation of the distal part of the leg below the knee inward, resulting in a bowlegged appearance. Valgus means deviation of the distal part of the leg below the knee outward, resulting in a knock-kneed appearance.

Although the implants, systems, and methods described and contemplated herein are disclosed in the context of a tibial bone of knee joint to streamline the present disclosure, it will be understood that the basic concepts inherent in the implants, systems, and methods described and contemplated herein can be adapted for utilization in any load bearing joint that has multiple condyles or articulation surfaces that are separated from each other by at least one non-load bearing area that may be adapted to bear at least a portion of a compression load force.

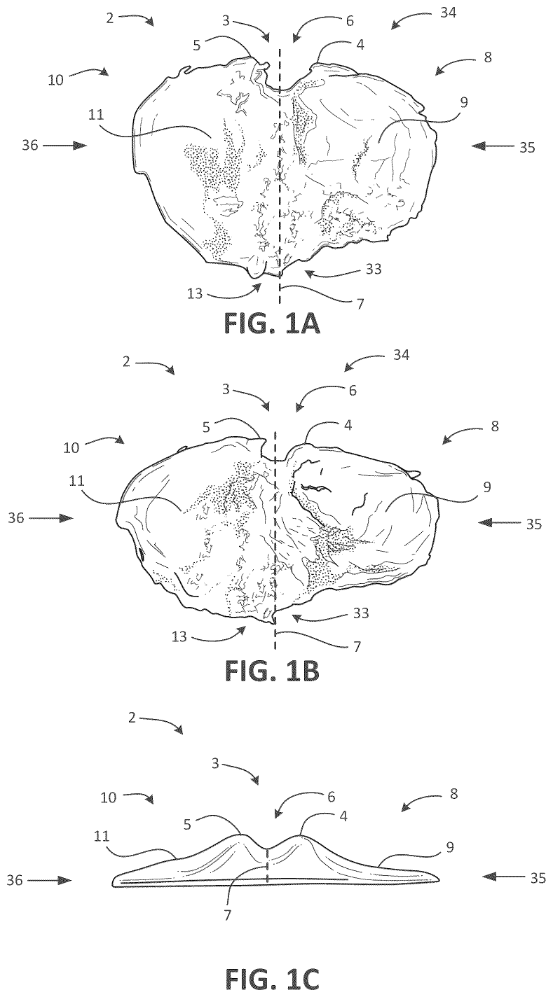

As shown in FIGS. 1A-1C, a tibial plateau 2 (shown resected from a tibial bone or tibia 1 in FIGS. 1A-1C) may include a proximal tibial articulating surface of the tibia 1 that defines an intercondyloid eminence 3 comprising a medial tibial eminence 4 and a lateral tibial eminence 5. The intercondyloid eminence 3 may be located intermediate a medial tibial condyle 8 comprising a medial tibial articular surface 9 on a medial side 35 of the tibial plateau 2, and a lateral tibial condyle 10 comprising a lateral tibial articular surface 11 on a lateral side 36 of the tibial plateau 2.

As defined herein, a “Goal Line tunnel” 6 of the tibial plateau 2 may comprise the intercondyloid valley or trough that is positioned intermediate the medial tibial eminence 4 and the lateral tibial eminence 5, and extends antero-posteriorly along the tibial plateau 2 from an anterior side 33 to a posterior side 34 of the tibial plateau 2. The Goal Line tunnel 6 is typically a non-weight bearing area of the tibial plateau 2 that is reserved for receiving the anterior cruciate ligament (ACL) therein as the knee joint progresses toward flexion.

As defined herein, a “Goal Line” 7 may traverse along a bottom surface of the Goal Line tunnel 6 and extend generally (but not solely) anteriorly-posteriorly between the medial tibial eminence 4 and the lateral tibial eminence 5 of the tibia 1. In some embodiments, the Goal Line 7 may be defined by a point near the medial ⅓ of a tibial tuberosity 14 of the tibia 1 that extends to a point near the center of the ACL insertion site or anterior aspect 13 on the Goal Line tunnel 6. The Goal Line 7 may thus extend through the “valley” or Goal Line tunnel 6 that is formed between the medial tibial eminence 4 and the lateral tibial eminence 5 on the tibial plateau 2. The Goal Line 7 may generally define the mechanical axis 12 or weight bearing center of the tibia 1 that extends anterior-to-posterior through the Goal Line tunnel 6. The Goal Line 7 may directly align with the mechanical axis of an ankle joint below the knee joint to achieve balanced weight bearing functionality.

As defined herein, a “Sulcus Line” may be a 3D line that is located along at least a portion of a trochlear groove 21 of a femoral bone intermediate a medial femoral condyle 17 having a medial femoral articular surface 18, and a lateral femoral condyle 19 having a lateral femoral articular surface 20. The Sulcus Line may extend generally (but not solely) anteriorly-posteriorly between the medial and lateral condyles of the femur 16 and may comprise a curved shape along at least a portion of the trochlear groove 21, which is curved.

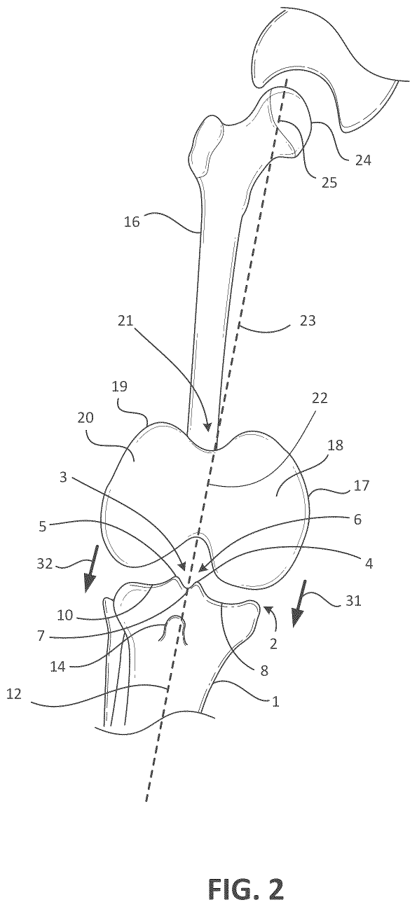

As defined herein, a Whiteside's Line 22 (e.g., see FIGS. 2-4) of a femur 16 may be defined by a line that intersects at least two points located along the trochlear groove 21 or Sulcus Line of the femur 16. The Whiteside's Line 22 may also be referred to as the Antero-Posterior Axis (APA) of the femur 16. The Whiteside's Line 22 may generally define a mechanical axis 23 or weight bearing center of the femur 16 that extends antero-posteriorly with respect to the trochlear groove 21 of the femur 16. The Whiteside's Line 22 may be directly aligned with a center 25 of rotation of a femoral head 24 of the femur 16 (e.g., see FIG. 2) to achieve balanced weight bearing functionality for the knee joint. Thus, the mechanical axis of the ankle joint, the Goal Line 7, the Whiteside's Line 22, and the center 25 of rotation for the femoral head 24 may all preferably align with each other to achieve a balanced knee joint, as shown in FIG. 2. In this manner, a first compression load force 31 transmitted across the medial condyles of the knee joint, and a second compression load force 32 transmitted across the lateral condyles of the knee joint may be in balance with each other when the knee joint is under load.

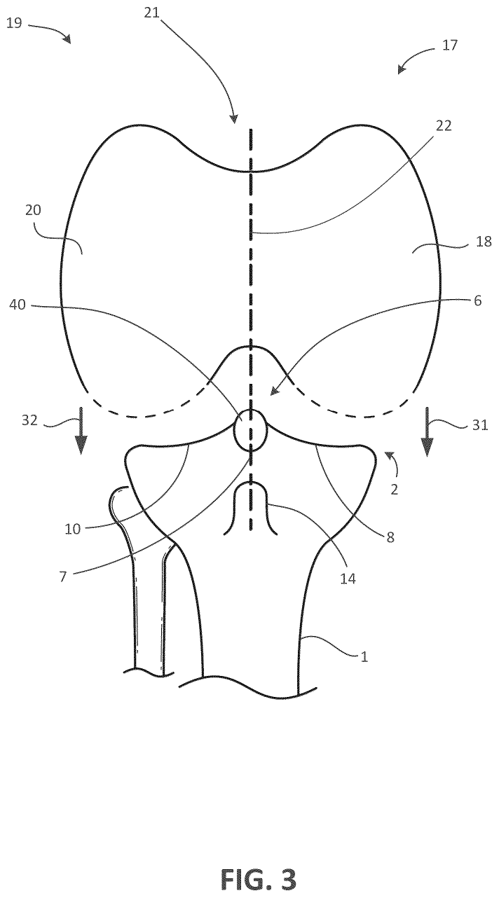

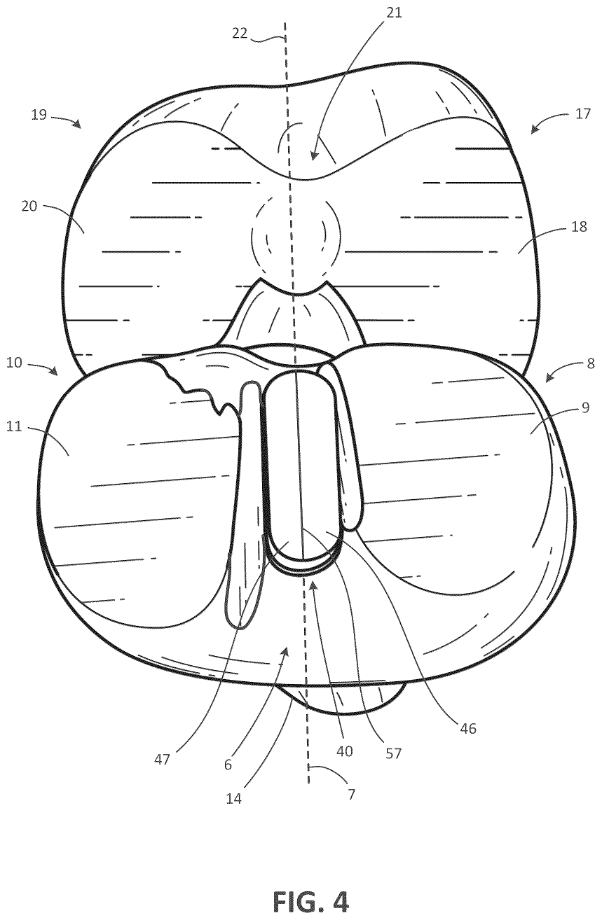

It has been discovered that, for at least some knee joint pathologies, it may be advantageous to utilize the non-weight bearing region of the Goal Line tunnel 6 area to share the compression load forces that are typically imparted on the tibia 1 by the femur 16 through the medial and lateral condyles. For example, some patients may have pathophysiologies that will likely require an invasive Total Knee Arthroplasty (TKA) procedure at some point in the future, assuming these pathophysiologies continue to progress over time. However, the need for a future TKA procedure may be delayed even further (or entirely prevented) by the devices, systems, and methods described herein, which exploit the non-weight bearing region of the Goal Line tunnel 6 area in order to share at least a portion of the compression load forces that are normally transmitted across the condyles of the knee joint. For example, FIGS. 3 and 4 show how an intra-eminence implant, load-sharing implant, bench implant, tibial implant, or implant 40 may be placed within the Goal Line tunnel 6 area of the tibia 1 to receive at least a portion of the compression load forces from the femur 16 for transmission through the Goal Line tunnel 6 area and into the tibia 1. In this manner, the compression load forces that would normally be transmitted across the condyles of the knee joint are now reduced in proportion to the compression load forces that are channeled through the implant 40 to the Goal Line tunnel 6 area of the tibia 1. Thus, an arthritic knee joint that is progressing toward a TKA procedure may be given additional support and relief to further delay (or entirely prevent) the need for an invasive TKA procedure.

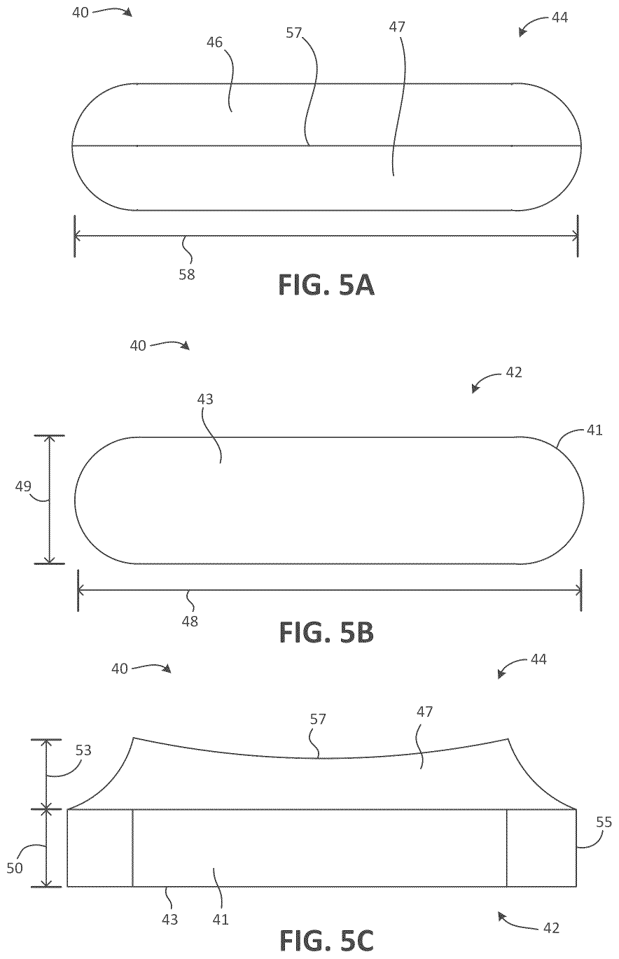

FIGS. 5A-5C illustrate various views of the implant 40 from FIG. 4, according to one embodiment of the present disclosure. Specifically, FIG. 1A shows a top view of the implant 40, FIG. 1B shows a bottom view of the implant 40, and FIG. 1C shows a side view of the implant 40.

In some embodiments, the implant 40 may include an elongate body 41 having a length 48, a width 49, a height 50, an inferior side 42 comprising a bone-facing surface 43, a superior side 44 comprising at least one implant articular surface, and a side wall 55 positioned intermediate the inferior side 42 and the superior side 44 of the implant 40.

In some embodiments, the bone-facing surface 43 may be positioned on the inferior side 42 of the implant 40 and configured to couple within or to the Goal Line tunnel 6 area of the tibia 1.

In some embodiments, the implant 40 may be shaped to be at least partially received within the Goal Line tunnel 6 of the tibia 1.

In some embodiments, the implant 40 or elongate body 41 may be at least partially positionable within the Goal Line tunnel 6 and the bone-facing surface 43 of the elongate body 41 may be couplable therein to secure the implant 40 to the tibia 1.

In some embodiments, the implant 40 or elongate body 41 may be positionable in at least an anterior aspect 13 of the Goal Line tunnel 6. However, it will be understood that the implant 40 or elongate body 41 may be positionable anywhere within or proximate the Goal Line tunnel 6.

In some embodiments, the length 48 of the implant 40 or elongate body 41 may be oriented antero-posteriorly along the Goal Line 7 located or defined within the Goal Line tunnel 6.

In some embodiments, the width 49 of the implant 40 or elongate body 41 may be configured to cover no more than 50% of a width of a condyle of the tibia 1.

In some embodiments, the width 49 of the implant 40 or elongate body 41 may be configured to cover no more than 40% of a width of a condyle of the tibia 1.

In some embodiments, the width 49 of the implant 40 or elongate body 41 may be configured to cover no more than 30% of a width of a condyle of the tibia 1.

In some embodiments, the width 49 of the implant 40 or elongate body 41 may be configured to cover no more than 25% of a width of a condyle of the tibia 1.

In some embodiments, the width 49 of the implant 40 or elongate body 41 may be configured to cover no more than 20% of a width of a condyle of the tibia 1.

In some embodiments, the width 49 of the implant 40 or elongate body 41 may be configured to cover no more than 15% of a width of a condyle of the tibia 1.

In some embodiments, the width 49 of the implant 40 or elongate body 41 may be configured to cover no more than 10% of a width of a condyle of the tibia 1.

In some embodiments, the width 49 of the implant 40 or elongate body 41 may be configured to cover no more than 5% of a width of a condyle of the tibia 1.

In some embodiments, the width 49 of the implant 40 or elongate body 41 may be configured to cover no width, or substantially no width, of a condyle of the tibia 1.

In some embodiments, a majority of the width 49 of the implant 40 or elongate body 41 may be positionable within the Goal Line tunnel 6.

In some embodiments, a majority of the length 48 of the implant 40 or elongate body 41 may be positionable along the Goal Line 7 located or defined within the Goal Line tunnel 6.

In some embodiments, the at least one implant articular surface may be positioned on the superior side 44 of the implant 40, opposite the bone-facing surface 43.

In some embodiments, the at least one implant articular surface may be configured to receive a compression load force from at least one femoral articular surface engaged with the at least one implant articular surface.

In some embodiments, the bone-facing surface 43 coupled within the Goal Line tunnel 6 may be configured to transmit the compression load force received from the femur 16 to the tibia 1 along the Goal Line 7.

In some embodiments, the implant 40 may be fixedly secured to the Goal Line tunnel 6 along, or with respect to, the Goal Line 7 via any suitable method or structure (e.g., via one or more bone screws, spikes, keels, or other bone fixation elements, etc.).

In some embodiments, the bone-facing surface 43 of the implant 40 may be porous and/or otherwise designed for osseointegration with the adjoining tibial bone (e.g., via a nano-coating, a plasma coating, or any bone-growth stimulating material, coating, bioactive material, pharmaceutical, hormone, etc.).

In some embodiments, the implant 40 may comprise separate bone-facing and articular components. In these embodiments, the bone-facing component may be secured to the bone, and the articular component may then be secured to the bone-facing component. Any techniques or configurations known in the design and implantation of tibial TKA components may be utilized in these embodiments. For example, in some embodiments the articular component may be slidably secured to the bone-facing component, such that the articular surface is able to slide in the anterior-posterior direction relative to the bone-facing component coupled to the tibia 1 and/or slidably translate along the Goal Line 7. This embodiment may act similar to a mobile bearing tibial component, but may be installed in the knee joint with a much less invasive surgical technique that can preserve all/most of the natural condyles of the tibia 1.

In some embodiments, at least one of: a height 53, a shape, and/or a position of the at least one implant articular surface (relative to the at least one femoral articular surface engaged therewith) may be selectable to receive any desired percentage or percentage range of the compression load force for transmission to the tibia 1 for any position or range of positions of the knee joint between full flexion and full extension.

In some embodiments, the desired percentage or percentage range may be greater than or equal to 0% and less than or equal to 100%.

In some embodiments, the desired percentage or percentage range may be between 10% to 90%.

In some embodiments, the desired percentage or percentage range may be between 20% to 80%.

In some embodiments, the desired percentage or percentage range may be between 30% to 70%.

In some embodiments, the desired percentage or percentage range may be between 40% to 60%.

In some embodiments, the desired percentage or percentage range may be about 50%.

However, it will be understood that the desired percentage or percentage range may be any value or range of values from 0% and 100%, which may be achieved by varying at least one of: a height 53, a shape, and/or a position of the at least one implant articular surface relative to the at least one femoral articular surface that is engaged therewith.

In some embodiments, the desired percentage or percentage range may be configured to increase as the knee joint moves from flexion toward extension. This may relieve the load ordinarily experienced by the anterior aspects of the condyles of knee joint (which occurs mostly toward extension). However, it will also be understood that the desired percentage or percentage range can be configured to decrease as the knee joint moves from flexion toward extension and/or vary in magnitude in any manner and over any desired range(s) of motion, in at least some embodiments.

In some embodiments, the implant 40 may be positioned and/or shaped to make contact with the femur 16 only during extension, or only over a desired range of motion for the knee joint between flexion and extension.

In some embodiments, the implant 40 may be positioned, contoured, and/or shaped to contact the femur 16 throughout a desired range of motion between flexion and extension of the knee joint to achieve a desired constant or varying percentage or percentage range of the compression load force for transmission to the tibia 1 over the desired range of motion.

In some embodiments, the at least one implant articular surface of the implant 40 may be shaped to engage the surfaces of the medial and lateral sides of the femur 16 near the Sulcus Line or the Whiteside's Line 22 over at least some range of motion for the knee joint. Alternatively, the at least one implant articular surface of the implant 40 may be shaped to engage one or more surfaces on an artificial femoral component (not shown) installed on the femur 16 and shaped to engage the at least one implant articular surface of the implant 40.

In some embodiments, the at least one implant articular surface may comprise a medial implant articular surface 46 and a lateral implant articular surface 47, and the at least one femoral articular surface may comprise the medial femoral articular surface 18 and the lateral femoral articular surface 20. The medial implant articular surface 46 may be configured to receive the first compression load force 31 from the medial femoral articular surface 18 that is engaged with the medial implant articular surface 46, and the lateral implant articular surface 47 may be configured to receive the second compression load force 32 from the lateral femoral articular surface 20 that is engaged with the lateral implant articular surface 47.

In some embodiments, the medial implant articular surface 46 and the lateral implant articular surface 47 may comprise at least partially concave surfaces that may slope medially-laterally, anteriorly-posteriorly, or not at all.

In some embodiments, the medial femoral articular surface 18 and the lateral femoral articular surface 20 may comprise at least partially convex surfaces that may slope medially-laterally, anteriorly-posteriorly, or not at all.

In some embodiments, the implant 40 may comprise an implant ridge 57 positioned intermediate the medial implant articular surface 46 and the lateral implant articular surface 47.

In some embodiments, the implant ridge 57 may comprise one or more concave curvatures along the length 48 of the implant 40. However, it will also be understood that the implant ridge 57 may comprise one or more convex curvatures (or no curvature at all) over all or any portion of the length 58 of the implant ridge 57.

In some embodiments, any portion of the implant ridge 57 may slope medially/laterally, anteriorly/posteriorly, or not at all.

In some embodiments, the shape of the implant ridge 57 may be selectable to comprise one or more sharper or pointed ridges that may act to limit medial-lateral rotational motion for the knee joint.

In some embodiments, the shape of the implant ridge 57 may be selectable to comprise one or more smoother ridge shapes that may allow for varying degrees of medial-lateral rotational motion for the knee joint.

In some embodiments, the implant ridge 57 may be centrally located (or approximately centrally located) on the superior side 44 of the implant 40 and/or generally aligned antero-posteriorly with at least one of the Whiteside's Line 22, the Sulcus Line, the Goal Line 7, and/or the Goal Line tunnel 6.

In some embodiments, the at least one implant articular surface of the implant 40 may be patient-specific and/or constructed based on imaging data, such as a CT scan of the patient's knee.

In some embodiments, the at least one implant articular surface of the implant 40 may be formed of any suitable material, such as polyethylene (e.g., UHMWPE, etc.), any metal, any metal alloy, etc.

In some embodiments, a method for installing an implant 40 within a Goal Line tunnel 6 of a tibial plateau 2 on a tibia 1 may comprise: (1) exposing the tibial plateau 2 at a surgical site proximate the Goal Line tunnel 6; (2) inserting the implant 40 through the surgical site and into the Goal Line tunnel 6 of the tibial plateau 2; and (3) coupling a bone-facing surface 43 of the implant 40 to the Goal Line tunnel 6 to secure the implant 40 to the tibia 1.

In some embodiments, the method may also comprise orienting a length 48 of the implant 40 along a Goal Line 7 that is located or defined within the Goal Line tunnel 6.

In some embodiments, the tibial plateau 2 may be exposed at the surgical site via a minimally invasive surgical technique, and a patellar tendon of the knee joint may be pulled aside to reveal the anterior aspect 13 of the of the Goal Line tunnel 6.

In some embodiments, the method may also comprise preparing the Goal Line tunnel 6 to receive the implant 40 therein. For example, the Goal Line tunnel 6 may be prepared by excising an anterior cruciate ligament that is coupled to the anterior aspect 13 of the Goal Line tunnel 6, to reveal the Goal Line tunnel 6 for insertion therein. Moreover, the Goal Line tunnel 6 may be additionally prepared by widening the Goal Line tunnel 6 to receive the implant 40 therein (e.g., via resecting, rasping, broaching, etc., to remove/shape any portion of the Goal Line tunnel 6, or any other part of the tibial plateau 2, and enable reception of the implant 40 therein).

In some embodiments, the tibial implant may be inserted into the Goal Line tunnel from an anterior-to-posterior direction.

In some embodiments, the tibial implant may be inserted into the Goal Line tunnel from a posterior-to-anterior direction.

In some embodiments, a method of distributing a compression load force to a Goal Line tunnel 6 area of a tibia 1 via an implant 40 placed therein may comprise: (1) receiving at least a portion of the compression load force at one or more implant articular surfaces positioned on a superior side 44 of the implant 40; (2) transmitting the at least a portion of the compression load force to a bone-facing surface 43 positioned on an inferior side 42 of the implant 40 coupled within the Goal Line tunnel 6; and (3) distributing the at least a portion of the compression load force through the Goal Line tunnel 6 to the tibia 1 from the implant 40 coupled within the Goal Line tunnel 6.

In some embodiments of the method, the at least a portion of the compression load force may be distributed through the Goal Line tunnel 6 to the tibia 1 along a Goal Line 7 located or defined within the Goal Line tunnel 6.

In some embodiments, the method may also comprise selecting at least one of: a height 53, a shape, and a position of the one or more implant articular surfaces (relative to one or more femoral articular surfaces engaged therewith), to receive a desired percentage or percentage range of the compression load force for transmission to the tibia 1 through the Goal Line tunnel 6.

In some embodiments of the method, the desired percentage or percentage range may be greater than or equal to 0% and less than or equal to 100%.

In some embodiments of the method, the desired percentage or percentage range may be between 10% to 90%.

In some embodiments, the desired percentage or percentage range may be between 20% to 80%.

In some embodiments of the method, the desired percentage or percentage range may be between 30% to 70%.

In some embodiments of the method, the desired percentage or percentage range may be between 40% to 60%.

In some embodiments of the method, the desired percentage or percentage range may be about 50%.

However, it will be understood that the desired percentage or percentage range may be any value or range of values from 0% and 100%, which may be achieved by varying at least one of: a height 53, a shape, and/or a position of the at least one implant articular surface relative to the at least one femoral articular surface that is engaged therewith.

In some embodiments of the method, the desired percentage or percentage range may be configured to increase as the knee joint moves from flexion toward extension. This may relieve the load ordinarily experienced by the anterior aspects of the condyles of knee joint (which occurs mostly toward extension). However, it will also be understood that the desired percentage or percentage range can be configured to decrease as the knee joint moves from flexion toward extension and/or vary in magnitude in any manner and over any desired range(s) of motion, in at least some embodiments.

Any procedures or methods disclosed herein may comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the present disclosure. This method of disclosure, however, is not to be interpreted as reflecting an intention that any embodiment requires more features than those expressly recited in that embodiment. Rather, inventive aspects lie in a combination of fewer than all features of any single foregoing disclosed embodiment.

Recitation of the term “first” with respect to a feature or element does not necessarily imply the existence of a second or additional such feature or element. Elements recited in means-plus-function format are intended to be construed in accordance with 35 U.S.C. § 112. It will be apparent to those having skill in the art that changes may be made to the details of the above-described embodiments without departing from the underlying principles set forth herein.

The phrases “connected to”, “coupled to”, “engaged with”, and “in communication with” may refer to any form of interaction between two or more entities, including mechanical, electrical, magnetic, electromagnetic, fluid, and thermal interaction. Two components may be functionally coupled to each other even though they are not in direct contact with each other. The term “coupled” can include components that are coupled to each other via integral formation, components that are removably and/or non-removably coupled with each other, components that are functionally coupled to each other through one or more intermediary components, etc. The term “abutting” refers to items that may be in direct physical contact with each other, although the items may not necessarily be attached together. The phrase “fluid communication” refers to two or more features that are connected such that a fluid within one feature is able to pass into another feature. As defined herein the term “substantially” means within +/−20% of a target value, measurement, or desired characteristic.

While specific embodiments and applications of the present disclosure have been illustrated and described, it is to be understood that the scope of the present disclosure is not limited to the precise configurations and components disclosed herein. Various modifications, changes, and variations which will be apparent to those skilled in the art may be made in the arrangement, operation, and details of the instruments and methods disclosed herein.

What is claimed is:

1. An implant comprising:
a bone-facing surface positioned on an inferior side of the implant, the bone-facing surface configured to couple within a Goal Line tunnel of a tibial bone; and
at least one implant articular surface positioned on a superior side of the implant opposite the bone-facing surface;
wherein:
the implant is shaped to be at least partially received within the Goal Line tunnel of the tibial bone;
a length of the implant is oriented antero-posteriorly along a Goal Line located within the Goal Line tunnel;
the at least one implant articular surface is configured to receive a compression load force from at least one femoral articular surface engaged with the at least one implant articular surface; and
the bone-facing surface coupled within the Goal Line tunnel is configured to transmit the compression load force to the tibial bone along the Goal Line.

2. The implant of claim 1, wherein a majority of a width of the implant is positionable within the Goal Line tunnel.

3. The implant of claim 1, wherein a majority of the length of the implant is positionable along the Goal Line within the Goal Line tunnel.

4. The implant of claim 1, wherein at least one of:
a height;
a shape; and
a position of the at least one implant articular surface, relative to the at least one femoral articular surface, is selectable to receive a desired percentage range of the compression load force for transmission to the tibial bone.

5. The implant of claim 4, wherein the desired percentage range is 10% to 90%.

6. The implant of claim 4, wherein the desired percentage range is configured to increase from flexion toward extension of a knee joint comprising the implant.

7. The implant of claim 1, wherein:
the at least one implant articular surface comprises:
a medial implant articular surface; and
a lateral implant articular surface; and
the at least one femoral articular surface comprises:
a medial femoral articular surface; and
a lateral femoral articular surface;
wherein:
the medial implant articular surface is configured to receive a first compression load force from the medial femoral articular surface that is engaged with the medial implant articular surface; and
the lateral implant articular surface is configured to receive a second compression load force from the lateral femoral articular surface that is engaged with the lateral implant articular surface.

8. The implant of claim 7, wherein:
the medial implant articular surface and the lateral implant articular surface comprise concave surfaces; and
the medial femoral articular surface and the lateral femoral articular surface comprise convex surfaces.

9. The implant of claim 7, further comprising an implant ridge positioned intermediate the medial implant articular surface and the lateral implant articular surface.

10. The implant of claim 9, wherein the implant ridge comprises a concave curvature along the length of the implant.

11. A tibial implant comprising:
an elongate body comprising:
a length;
a width;
a bone-facing surface positioned on an inferior side of the elongate body, the bone-facing surface configured to couple within a Goal Line tunnel of a tibial bone; and
at least one implant articular surface positioned on a superior side of the elongate body opposite the bone-facing surface;
wherein:
the elongate body is positionable within the Goal Line tunnel and the bone-facing surface of the elongate body is couplable therein to secure the tibial implant to the tibial bone;
the length of the elongate body is oriented antero-posteriorly along a Goal Line defined within the Goal Line tunnel; and
the width of the elongate body is configured to cover no more than 20% of a width of a condyle of the tibial bone.

12. The tibial implant of claim 11, wherein:
the at least one implant articular surface comprises:
a medial implant articular surface; and
a lateral implant articular surface; and
the at least one femoral articular surface comprises:
a medial femoral articular surface; and
a lateral femoral articular surface;
wherein:
the medial implant articular surface is configured to receive a first compression load force from the medial femoral articular surface that is engaged with the medial implant articular surface; and
the lateral implant articular surface is configured to receive a second compression load force from the lateral femoral articular surface that is engaged with the lateral implant articular surface.

13. A method for installing a tibial implant within a Goal Line tunnel formed in a tibial plateau of a tibial bone, the method comprising:
exposing the tibial plateau at a surgical site proximate the Goal Line Tunnel;
inserting the tibial implant through the surgical site and into the Goal Line tunnel of the tibial plateau; and
coupling a bone-facing surface of the tibial implant to the Goal Line tunnel to secure the tibial implant to the tibial bone.

14. The method of claim 13, further comprising:
orienting a length of the tibial implant along a Goal Line located within the Goal Line tunnel.

15. The method of claim 13, further comprising:
preparing the Goal Line tunnel to receive the tibial implant therein.

16. The method of claim 15, wherein preparing the Goal Line tunnel to receive the tibial implant therein comprises:
excising an anterior cruciate ligament that is coupled to an anterior aspect of the Goal Line tunnel.

17. The method of claim 15, wherein preparing the Goal Line tunnel to receive the tibial implant therein comprises:
widening the Goal Line tunnel to receive the tibial implant therein.

18. The method of claim 13, wherein the tibial implant is inserted into the Goal Line tunnel from an anterior-to-posterior direction.

19. The method of claim 13, wherein the tibial implant is inserted into the Goal Line tunnel from a posterior-to-anterior direction.

20. A method of distributing a compression load force to a Goal Line tunnel of a tibial bone via an implant placed therein, the method comprising:
receiving at least a portion of the compression load force at one or more implant articular surfaces positioned on a superior side of the implant;
transmitting the at least a portion of the compression load force to a bone-facing surface positioned on an inferior side of the implant that is coupled within the Goal Line tunnel; and
distributing the at least a portion of the compression load force through the Goal Line tunnel to the tibial bone from the implant that is coupled within the Goal Line tunnel.

21. The method of claim 20, wherein the at least a portion of the compression load force is distributed through the Goal Line tunnel to the tibial bone along a Goal Line located within the Goal Line tunnel.

22. The method of claim 20, further comprising:
selecting at least one of:
a height;
a shape; and
a position of the one or more implant articular surfaces, relative to one or more femoral articular surfaces, to receive a desired percentage range of the compression load force for transmission to the tibial bone through the Goal Line tunnel.

23. The method of claim 22, wherein the desired percentage range is 10% to 90%.

24. The method of claim 22, wherein the desired percentage range is configured to increase from flexion toward extension of a knee joint that comprises the implant.
